(19) United States
(12) Patent Application Publication
(10) Pub. No.: US 2009/0234255 A1
(43) Pub. Date: Sep. 17, 2009

(54) ERGONOMIC DEVICE FOR ADMINISTERING CARDIO-PULMONARY RESUSCITATION

(76) Inventor: Thomas W. Votel, St. Paul, MN (US)

Correspondence Address: Wm. Larry Alexander, Attorney at Law P.A., 1957 Oak Street, Hastings, MN, 55033-3542, US

(21) Appl. No.: 12/171,580

(22) Filed: Jul. 11, 2008

Related U.S. Application Data
(60) Provisional application No. 61/069,030, filed on Mar. 12, 2008.

(51) Int. Cl. A61H 31/00; A61B 17/00; A61B 19/00

(52) U.S. Cl. 601/41; 606/201; 128/898

(57) ABSTRACT

A CPR device depicted and disclosed provides for ergonomically administering CPR from the side of a patient, thereby reducing or eliminating the risk of injury and providing a means for maximally effective CPR. One embodiment is optionally attachable to a bed or gurney and provides a leveraged structure with a CPR pad, so that CPR can be performed from the side of the bed or gurney. Other devices may be portable, and can be unfolded to encircle a patient's chest and so that CPR can be more effectively administered from the patient's side by means of a lever or by means of compressed air-driven compression devices.

### CROSS-REFERENCES TO RELATED APPLICATIONS

This application claims priority under 35 U.S.C. §1.19 (e) to, and hereby incorporates by reference, U.S. Provisional Application No. 61/069,030, filed Mar. 12, 2008.

### BACKGROUND

Cardiopulmonary resuscitation (CPR) is administered to a patient whose heart has either stopped beating or is undergoing fibrillation and functions to restore blood flow from the patient's heart to the patient's brain and other organs. Normally, CPR, as chest compression, is performed by straddling the patient or leaning over the patient from the side to compress the chest with proximal palms to thereby provide rhythmic chest compressions. Both procedures expose the practitioner to risk of injury. Additionally, the patient is often in a bed or on a gurney on which a soft mattress is present. The mattress absorbs much of the force exerted on the patient's chest, thereby decreasing the effectiveness of the compression. Practitioners in these situations often straddle the patient on a soft surface, thereby risking injury to the practitioner's back or injury due to the practitioner falling from the bed or gurney while administering CPR. It is known that CPR administered from a patient's side is considerably less effective than CPR administered while straddling the patient. When a patient is disposed on a gurney, the underlying surface is firm; however, CPR must still be administered while either straddling the patient or from the patient's side. Either position is less than optimally effective and/or exposes the practitioner to injury as described above. Additionally, a practitioner's hands are not optimal instrumentalities for administering CPR to a patient's chest. In the situation where a patient is disposed on a mattress, a board or other stiff structure placed under the patient improves compression.

### SUMMARY

The CPR device of this invention provides an ergonomic method for administering chest compression that both safeguards a practitioner from injury and administers CPR effectively. The CPR device of this invention includes an optionally smooth, e.g., plastic, surface, which can be placed beneath the patient. In some embodiments, the surface is hinged to an upper component surface lying over the chest of the patient and having an effective, efficient plunger aligned over the heart so as to provide more efficient compression. This upper surface may have an extension permitting health-care workers to kneel or stand next to the patient and to use the leverage of this extension to effectively compress the chest. Chest compression using the CPR device of this invention, accordingly, is more efficient and can be accomplished without risk of injury to a health-care worker. Embodiments of this device may be folded neatly for easy storage. Other embodiments of the CPR device of this invention may include an elongated, retractable-extendable handle, providing leverage when extended during use, yet retracting for storage, so as to conserve space and fit beneath a bed or cart.

The CPR device of this invention may include a lever extending over the patient's chest and anchored to a bed rail, cart rail, or frame by a swivel-type receptacle having a flexible or pivoting hinge and receiving the lever at one end. The free end of the lever may be used to exert a pumping action across the patient's chest when administering CPR. Present on the lever may be a compression device adapted to the chest wall over the patient's heart. The lever may have a compression device, which can be adapted to the chest wall over the patient's heart. The compression device may be manufactured from inert material and may further include a controlling device regulating compression force applied to the patient. The lever may be restricted, or the amount of compression determined, by use of a compression gauge or the like.

The CPR device of this invention may be in the embodiment of a CPR kit comprising a flat surface, a lateral component, and a ventral component. The flat surface may provide a dorsally positioned base under the patient's chest. The lateral component provides elevation and a base for attaching a hinged lever. The ventral component covers the patient's chest and may have a compression component to conform to the area over the patient's heart. Optionally, the foregoing operating components may be unfolded to form the compression unit of this invention, then refolded into a small, easily stored kit.

### BRIEF DESCRIPTION OF THE FIGURES

FIG. 1 is a plan view depicting a first embodiment of a CPR device of this invention being used on a patient.

FIG. 2 is a side view of the CPR device of FIG. 1 being used on a patient.

FIG. 3 is another side view of the CPR device of FIG. 1 being used on a patient.

FIG. 4 is a cross-sectional view of one embodiment of an attachment member of the CPR device depicted in FIG. 1.

FIG. 5 is a side view of a second embodiment of the CPR device of this invention configured in a storage configuration.

FIG. 6 is a perspective view of the CPR embodiment depicted in FIG. 5 in a use configuration.

FIG. 7 is a perspective view of the CPR embodiment depicted in FIG. 5 in a storage configuration.

FIG. 8 is a perspective view of the CPR device depicted in FIG. 5 being used to administer CPR to a patient.

FIG. 9 is a side view of a third embodiment of the CPR device of this invention configured in a storage configuration.

FIG. 10 is a perspective view of the CPR device depicted in FIG. 9 in a use configuration.

FIG. 11 is a perspective view of the CPR device depicted in FIG. 9 being used to administer CPR to a patient.

FIG. 12 is a perspective view of the CPR device depicted in FIG. 9 in a storage configuration.

FIG. 13 is a side view of a fourth embodiment of the CPR device of this invention configured in a storage configuration.

FIG. 14 is a perspective view of the CPR device depicted in FIG. 13 in a use configuration.

FIG. 15 is a perspective view of the CPR device depicted in FIG. 13 being used to administer CPR to a patient.

FIG. 16 is a perspective view of the CPR device depicted in FIG. 13 in a storage configuration.

It is understood that the above-described figures are only illustrative of the present invention and are not contemplated to limit the scope thereof.

### DETAILED DESCRIPTION

Each of the additional features and methods disclosed herein may be utilized separately or in conjunction with other features and methods to provide improved devices of this invention and methods for making and using the same. Representative examples of the teachings of the present invention, which examples utilize many of these additional features and methods in conjunction, will now be described in detail with reference to the drawings. This detailed description is merely intended to teach a person of skill in the art further details for practicing preferred aspects of the present teachings and is not intended to limit the scope of the invention. Therefore, combinations of features and methods disclosed in the following detailed description may not be necessary to practice the invention in the broadest sense and are instead taught merely to particularly describe representative and preferred embodiments of the invention. Additionally, a person of ordinary skill in the art will appreciate that individual components shown on various embodiments of the present invention are interchangeable to some extent and may be added or interchanged on other embodiments without departing from the spirit and scope of this invention.

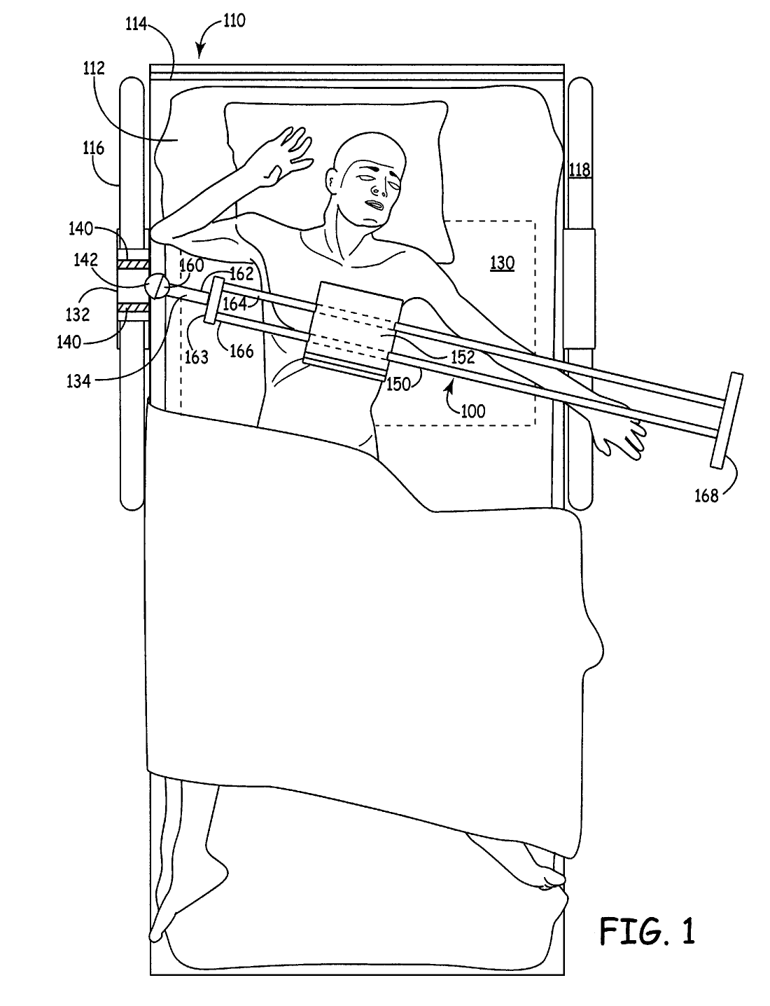

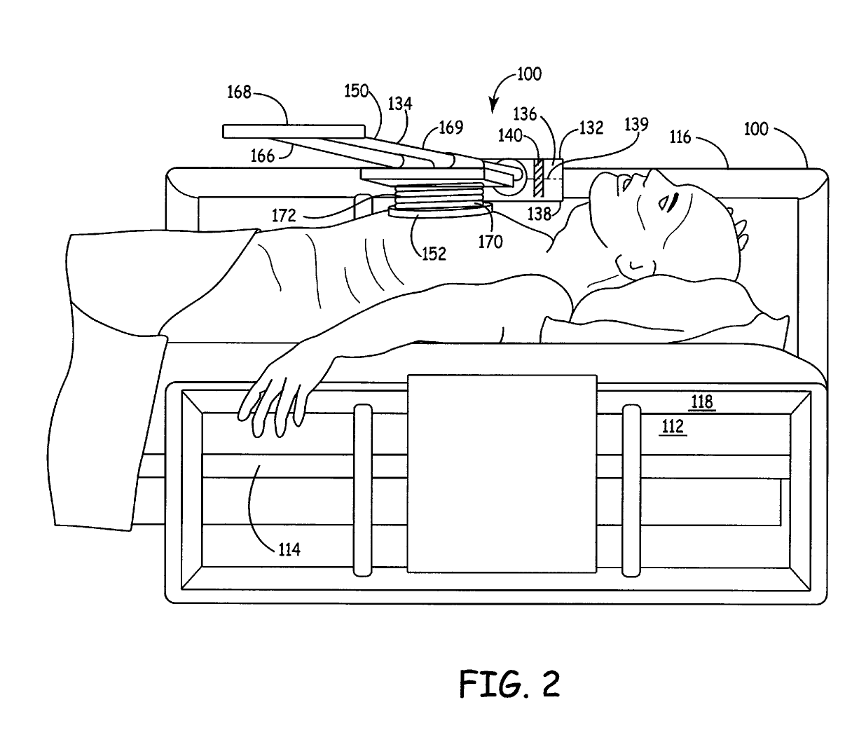

Referring to FIGS. 1-4, one embodiment of the cardio-pulmonary resuscitation (CPR) device of this invention is indicated generally at 100 and is mounted to a bed 110. The bed 110 includes a mattress 112 supported by a bed frame 114, as well as rails 116, 118. The rails 116, 118 may be raised or lowered as needed in a hospital environment. The bed 110 may be stationary or portable; indeed, a gurney modified as described below may be used in place of the bed 110.

The CPR device 100 depicted in FIGS. 1-3 includes a flat member (insert) 130, attachment member 132, and compression member 134.

The flat member 130 is operationally positioned beneath the chest of the patient when the CPR device 100 is being used. While a variety of materials may be used, the flat member 130 includes a synthetic resin such as polyethylene, polypropylene, or polycarbonate. However, a person of ordinary skill in the art will readily recognize that other polymers may be utilized as well. Accordingly, suitable polymers are disclosed and described in the Handbook of Plastics, Elastomers, and Composites, Third Edition, Charles A. Harper, Editor-in-Chief, McGraw-Hill, New York (1996), hereby incorporated by reference in its entirety. A person of ordinary skill in the art will further recognize that other materials could be used in manufacturing the flat member 130, including wood and metals such as aluminum and steel alloys. In one embodiment the flat member 130 is substantially impervious to liquids and can be cleaned and sanitized between uses. However, other embodiments include materials suitable for a single use, then discarded. Factors determining the materials used for the flat member 130 of this invention include cost, rigidity, weight, and reusability (or lack thereof).

The attachment (side) member 132 is depicted as attached to the rail 116 in FIGS. 1-3. In one embodiment, the attachment member 132 attaches reversibly to the rail 116. In another embodiment, the attachment member 132 is a permanent fixture of one or both of the rails 116, 118. Referring to FIG. 4, the attachment member 132 depicted is generally circular, following a cross sectional contour of the rails 116, 118 and includes attachment member halves 136, 138 connected at a hinge 139 and secured about the rails 116, 118 by a pair of clamp members 140. The clamp members 140 may be utilized to quickly attach at a desired location, then reversibly detach the attachment member 132 from one of the rails 116, 118. Also envisioned but not depicted, are embodiments wherein the compression member 134 reversibly snaps into a socket present on the rail and which the ball 160 thereof swivels when attached. Alternatively, an eccentric (swivel) clamp (not shown) could be used for reversible attachments of this invention. Also shown in FIG. 1, but not depicted in FIG. 4, is a socket 142, optionally attached to the attachment member 132. A person of ordinary skill in the art will readily recognize that a horizontally oriented hinge or pair of hinges arranged normally with respect to each other may also be present in other embodiments, wherein structure facilitating pivoting or rotation of the compression member 134 is present. Additionally a flexible material such as cloth, or a synthetic polymer (e.g., living hinge) may be used, optionally fixed to the rail by a hook and loop mechanism. Alternatively, anchoring structure may be present on the rails 116, 118, wherein the compression member 134 can be optionally reversibly attached. As discussed above briefly, the attachment member 132 may be adapted for a gurney by a person of ordinary skill in the art. For example, the present CPR device could be mounted to the undercarriage of a gurney, optionally reversibly so, to be available for use, optionally providing an insert for the compression member frame 150 (discussed below).

The compression member 134, in the embodiment shown, includes a compression member frame 150 and compression element 152. The frame 150, in turn, has a ball 160, a ball shaft 162, frame elements 163, 164, 166 and a handle 168. The ball 160 is disposed within the socket 142, the socket 142 being attached to the attachment member 132. Consequently, in the embodiment shown, the attachment member 132 and compression member 134 are unitary and functional when the attachment member 132 is attached to one of the rails 116, 118. The ball shaft 162 is attached to, and extends between, the ball 160 and the frame element 163. Alternate mechanisms for the ball and socket are described above. The frame element 163 and handle 168, in turn, are perpendicularly, or otherwise transversely, attached to the frame elements 164, 166. The compression member frame 150 may be formed from materials such as wood, steel alloys, aluminum, or synthetic resins such as polycarbonate, polyethylene, polypropylene, and the like, alternatives readily selected by a person of ordinary skill in the art from The Handbook of Plastics, Elastomers, and Composites (incorporated by reference above). In one embodiment, the compression member frame 150 has a degree of rigidity such that only a certain force can be applied to a patient's chest during use, forces in excess bending or flexing the compression member frame to thereby avoid injuring the patient. Alternatively or additionally, the amount of force applied to the patient during CPR may be governed or indicated by a device such as a compression gauge.

The compression element 152 as depicted is longitudinally adjustable along the compression member frame 150 by means of slots or bores in which the frame elements 164, 166 are reversibly disposed. While depicted as a pad with a lower, relatively planar, surface, the compression element 152 may also have an attached suction element 170 to “grip” the patient's chest during use and may be compressible, e.g., by the presence of a bellows 172. Suitable materials for the compression element 152 include natural and synthetic polymers such as rubber and foamed synthetic rubbers with the requisite amount of firmness and rigidity. Optionally, the compression element 152 includes electrode attachments (not shown) for cardioversion or defibrillation. Corresponding electrodes may optionally be present in the flat member 130. One suitable defibrillator pad and assembly is disclosed in U.S. Pat. No. 4,779,630 issued to Scharnberg et al. and in U.S. Pat. No. 4,998,536, issued to Scharnberg, each hereby incorporated by reference. However, a person of ordinary skill in the art will readily comprehend other arrangements for cardioversion and/or defibrillation.

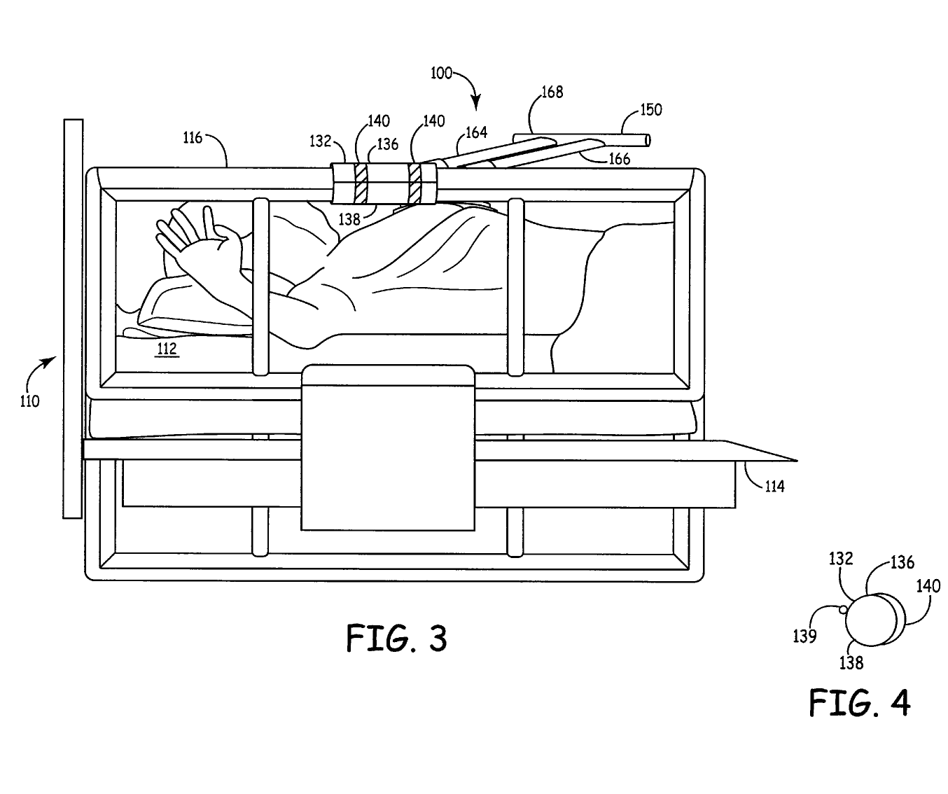

In use, the flat member 130 is placed beneath the patient's chest as shown in FIG. 3 and the remainder of the CPR device 100 is attached to the bed or gurney as described above. A health practitioner then grasps the handle 168, maneuvers the device such that the compression element 152 is situated above a left portion of the patient's chest (longitudinally adjusting the compression element 152 as needed), then exerts sufficient rhythmic, repeated force downwardly on the patient's chest until the person's heart resumes beating or until it is determined that further efforts are to be discontinued.

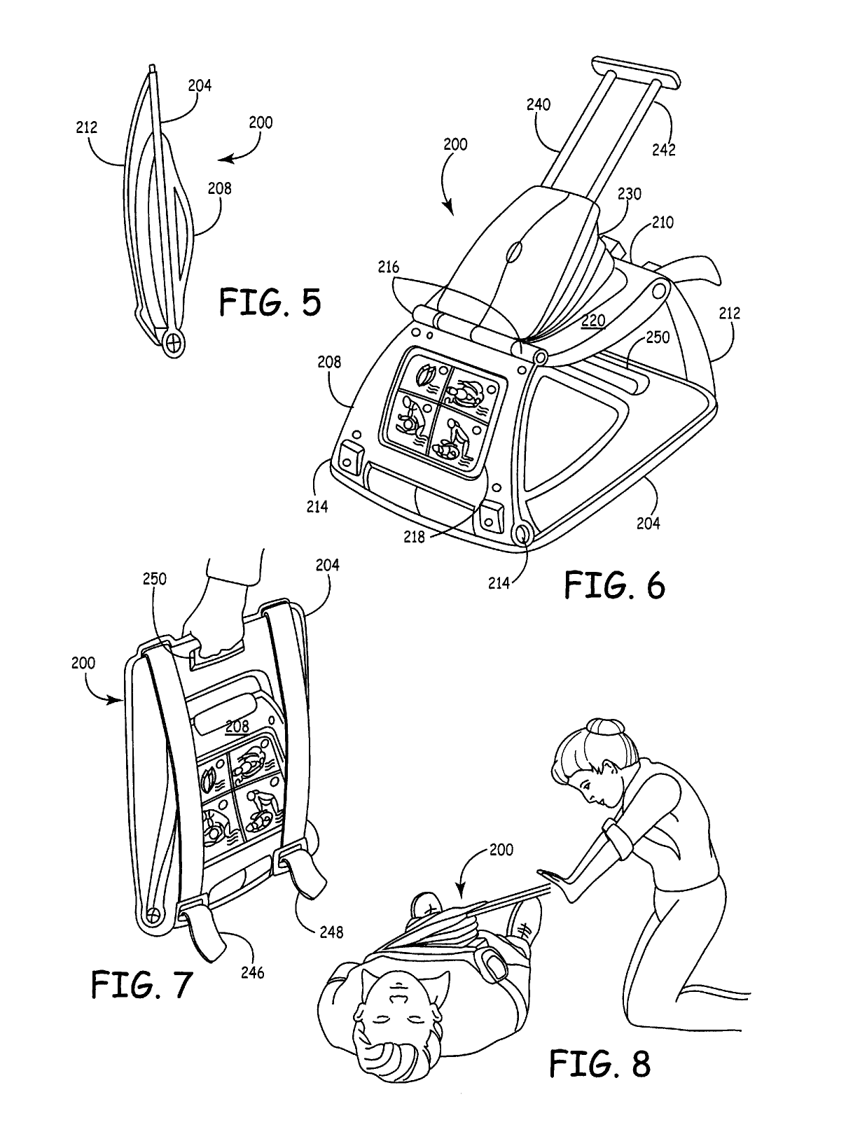

Referring to FIGS. 5-8 another embodiment of the CPR device of this invention is depicted generally at 200 and includes respective horizontal and vertical members 204, 208, a compression member 210, and strap assembly 212. The vertical member 208 is pivotally attached to the horizontal member 204 by means of one or more pivots or hinges 214 and also attached to the compression member by means of one or more hinges 216. The vertical member 208 may further include indicia 218, such as written and pictorial directions for use. The horizontal member 204 may be dimensioned to fit under a patient's chest, so as to provide a firm support to the patient during use. By way of illustration and not limitation, one suitable size for the horizontal member 204 is 24 inches×30 inches (60 cm×76 cm). Alternatively, the horizontal member may include a tacky surface to maintain the patient's thorax in place. Accordingly, the vertical member 208 is dimensioned so as to span the thickness (e.g., from dorsal to ventral surfaces) of a patient's chest region and may be optionally adjustable in doing so. The compression member 210, in turn, includes a compression pad 220, compression structure 230, and a framework, such as telescoping elements 240, 242 connected to a handle 244. The compression pad 220 may include materials such as those described above with respect to the compression element 152. The compression structure may be a bellows or other compressible material such as foam rubber or the like and optionally allows a specific amount of force to be exerted on a patient's chest before further compressing to protect the patent from injury. The telescoping elements 240, 242 are extended for use and retracted for storage, optionally including materials to govern the amount of force exerted on a patient's chest during use, wherein the telescoping elements 240, 242 bend or flex in response to a force beyond a certain magnitude.

For storage, the CPR device 200 is folded along hinges 214, 216, the handles retracted, and the stored CPR configuration is then secured by means of straps 246, 248 and carried using a handle 250. During use, the CPR device 200 is reconfigured from the storage configuration shown in FIG. 5, into the use configuration shown in FIG. 6. The horizontal member 204 is disposed beneath the patient's chest, then the straps 246, 248 secure the device about the patient, the telescoping elements 240, 242 are extended as shown in FIGS. 6, 8, and a practitioner begins CPR by grasping the handle 244 as depicted in FIG. 8. As shown in FIG. 8, the practitioner ergonomically, and considerably more effectively, administers CPR from the side of patent, rather than being forced to straddle the patient or by using the practitioner's arms and hands to directly compress the patient's chest.

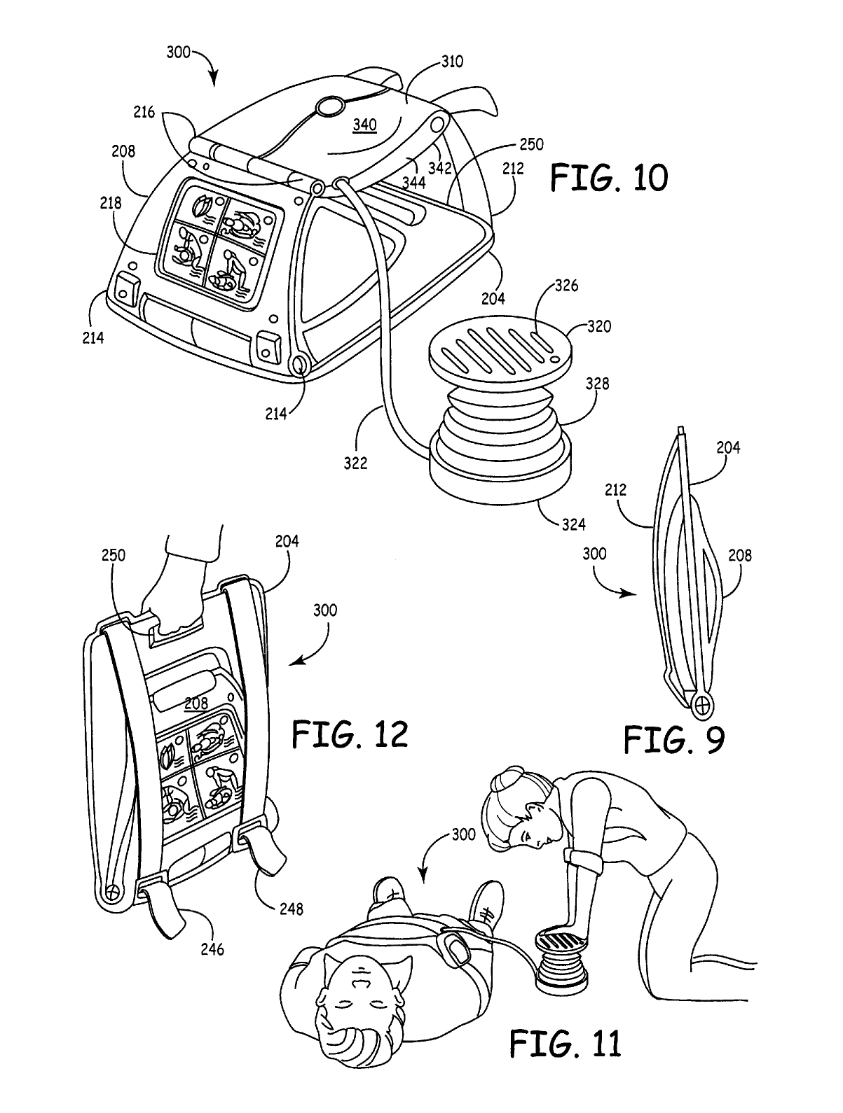

Referring to FIGS. 9-12, another embodiment of the CPR device of this invention is shown at 300, and, where numbered identically, includes structural elements present in the CPR device 200 (described above). The differences between the CPR device 300 and the CPR device 200 include a compression member 310 connected to a bellows 320 by means of a pneumatic line or hose 322. The bellows 320, in turn, includes disk elements 324, 326 and a bellows element 328 operably disposed between the disk elements 324, 326. Storage and use configurations of the CPR device 300 are depicted in respective FIGS. 12 and 10 and are achieved in a similar manner to those described above with respect to the CPR device 200. The compression member 310 may be airtight with generally rigid upper and lower elements 340, 342 and an inflatable member, e.g. bladder 344 or the like, disposed between the upper and lower elements 340, 342. In use, and as depicted in FIG. 11, the CPR device 300 is unfolded from the storage position shown in FIG. 12 into the use position depicted in FIG. 10, then secured about the patient as described above with respect to CPR device 200. Then and as depicted in FIG. 11, a practitioner compresses the air within the bellows 320, thereby conveying compressed air through the pneumatic line 322 and into the compression member 310. Once the compressed air enters the compression member 310, the bladder 344 is expanded to exert a force on the patient's chest, thereby performing CPR upon the patient. In this case again, CPR is ergonomically and more effectively administered from the patient's side when the CPR device 300 is used.

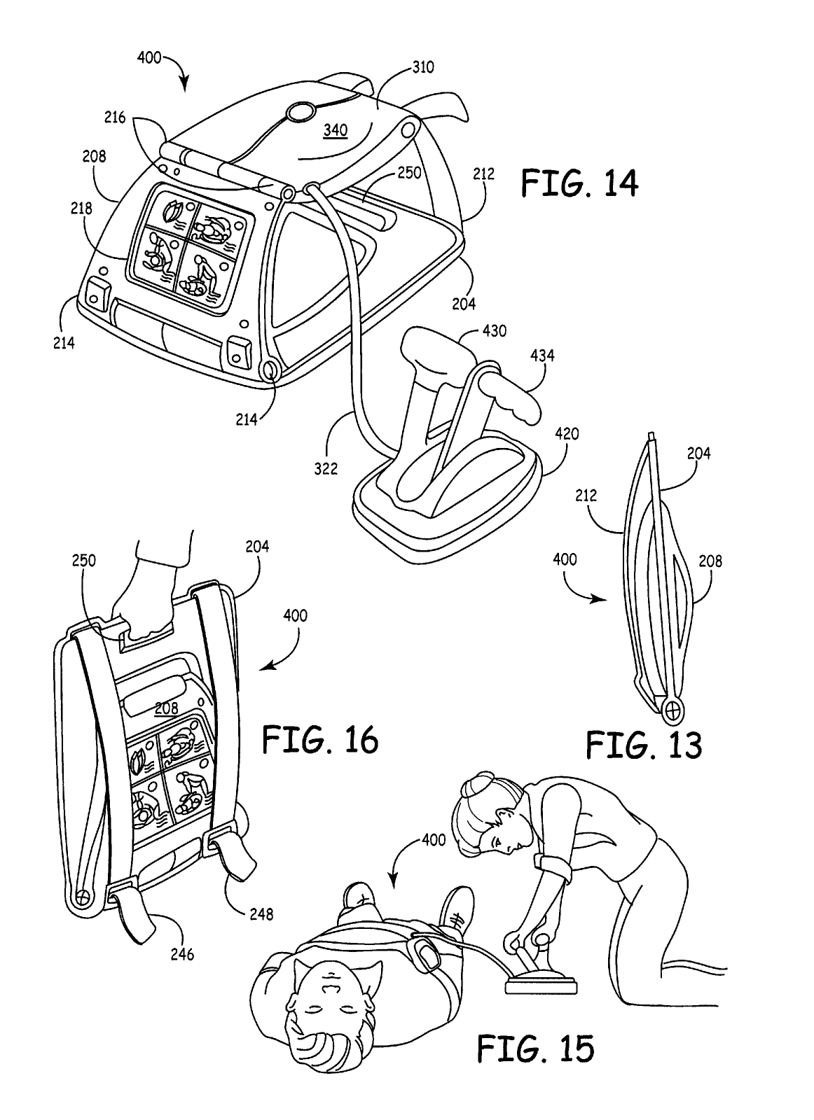

Referring now to FIGS. 13-16, another embodiment of the CPR device of this invention is shown at 400. Similar or substantially identical structural elements to those shown with respect to the CPR devices 200 and 300 are numbered identically. The CPR device 400 may be folded or unfolded between a storage configuration as shown in FIG. 16 and a use configuration as shown in FIG. 14 as described above with respect to the CPR device 200. Differences between the CPR device 400 and the CPR devices 200 and 300 include a compressor housing 420 with compressor handles 430, 434 and a compressor or air pump disposed within the compressor housing (compressor or air pump not shown). In use, the CPR device 400 is disposed about a patient as described above with respect to the CPR device 200 and as shown in FIG. 15. A practitioner then operates the compressor handles 430, 434 shown in FIG. 15 to administer CPR to the patient.

Similarly to the CPR device 100, any of the CPR devices 200, 300, and 400 may include electrodes for administering cardioversion or defibrillation and a person of ordinary skill in the art would readily appreciate how to select and dispose such structure therewithin.

Because numerous modifications of this invention may be made without departing from the spirit thereof, the scope of the invention is not to be limited to the embodiments illustrated and described. Rather, the scope of the invention is to be determined by the appended claims and their equivalents.

What is claimed is:

1. A method of administering CPR to a person disposed on a generally horizontal surface of a bed or a gurney by using a CPR device, the CPR device having an attachment member laterally attached to the bed or gurney, a compression member to rotatably extending from the attachment member and terminating in a handle distal to the attachment member, and a compression element attached to the compression member, the method comprising:
positioning said compression element over said person's chest;
grasping said handle; and
repeatedly applying a downward force on said handle, thereby exerting a rhythmic compression force on said person's chest.

2. The method of claim 1, further comprising disposing a generally rigid flat member beneath the person's chest.

3. The method of claim 1, in which said compression element is longitudinally adjusted on said compression member.

4. The method of claim 1, in which the compression element includes a suction element and in which the suction element grips the person's chest.

5. The method of claim 1, in which the compression element includes a bellows and in which the downward force is applied through the bellows.

6. The method of claim 1, in which the attachment member is reversibly attached to the bed or gurney.

7. The method of claim 1, in which the attachment member comprises a clamp and in which the clamp is attached to a side rail of the bed or gurney.

8. The method of claim 1, in which the attachment member is permanently attached to the bed or gurney and in which the compression member is reversibly attached to the attachment member.

9. A CPR device, comprising:
an attachment member for laterally attaching said attachment member to a bed or gurney;
a compression member rotatably attached to the attachment member and terminating in a handle; and
a compression element disposed on the compression member.

10. The CPR device of claim 9, further comprising a flat member disposable beneath a person's chest.

11. The CPR device of claim 9, in which the compression member is detachable from the attachment member.

12. The CPR device of claim 9, in which the attachment member includes a clamp.

13. The CPR device of claim 9, in which the attachment member includes a socket and in which the compression member comprises a ball accommodated by the socket.

14. The CPR device of claim 13, wherein compression member terminates in said ball.

15. The CPR device of claim 9, in which the attachment device is integral to a side rail of said bed or gurney.

16. The CPR device of claim 9, in which the attachment member is reversibly attached to the bed or gurney.

17. The CPR device of claim 9, in which a position of the compression element is longitudinally adjustable with respect to the compression member.

18. The CPR device of claim 9, in which the compression element comprises a suction element.

19. The CPR device of claim 9, in which the compression element comprises a bellows.

20. A bed or gurney in combination with the CPR device of claim 9, said CPR device attached thereto.

21. A method of manufacturing a CPR device, comprising providing an attachment member, a compression member and a compression element, the attachment member attaching to a bed or gurney, the compression member rotatably attachable to and extendable from the attachment member and including a handle distal from the attachment member, the compression element attachable to the compression member.

22. The method of claim 21, further comprising providing a generally rigid flat member.

23. The method of claim 21, in which the provided attachment member includes a clamp.

24. The method of claim 21, in which the provided attachment member includes a socket and in which the provided compression member includes a ball disposable in the socket and distal to the handle.

25. The method of claim 21, in which the provided compression element includes a bellows.

26. The method of claim 21, in which the provided compression element includes a suction element.

27. A method of retrofitting a bed or gurney with a CPR device, the CPR device having an attachment member attachable to the bed or gurney, a compression member rotatably extending from the attachment member and terminating in a handle distal to the attachment member, and a compression element attached to the compression member, the method comprising attaching said attachment member to the bed or gurney.

28. The method of claim 27, further comprising providing a flat member, said flat member disposable beneath a person's chest.

29. The method of claim 27, wherein the attachment member is attached to a side of the bed or gurney.

30. A portable CPR device, comprising:
a horizontal member;
a vertical member hingeably attached to the horizontal member; and
means for administering CPR attached to said vertical member,
said horizontal and vertical members folding together with said CPR administering means in a storage configuration and unfolding in a use configuration, said use configuration configured to enclose a chest of a person.

31. The portable CPR of claim 30, wherein the CPR administering means attaches to the vertical member with a strap.

32. The portable CPR of claim 30, in which the CPR administering means comprises a bellows and a handle.

33. The portable CPR of claim 32, in which the CPR administering means further comprises a pair of telescoping elements connecting to the handle and bellows.

34. The portable CPR of claim 30, in which the CPR administering means comprises a bellows, a bladder enclosed between upper and lower elements, and a pneumatic hose conveying compressed air from the bellows to the bladder.

35. The portable CPR of claim 30, in which the CPR administering means comprises a pump operated by a pair of handles, a bladder enclosed between upper and lower elements, and a pneumatic hose conveying compressed air between the pump and the bladder.

36. A method of manufacturing a portable CPR device, comprising:
hingeably attaching a vertical member to a horizontal member; and hingeably attaching a compression member to the vertical member.

37. The method of claim 36, wherein the compression member comprises a bellows, a pair of telescoping elements extending from the bellows, and a handle attached to the telescoping elements.

38. The method of claim 36, wherein the compression member comprises a bladder disposed between upper and lower generally rigid elements.

39. The method of claim 38, in which the compression member further comprises a bellows and a hose conveying compressed air from the bellows to the bladder.

40. The method of claim 38, in which the compression member further comprises a pump and a hose conveying compressed air from the pump to the bladder.
